(19) United States
(12) Patent Application Publication
(10) Pub. No.: US 2013/0103166 A1
(43) Pub. Date: Apr. 25, 2013

(54) Prosthetic Attachment Techniques

(71) Applicants: Nickolas Butler, San Luis Obispo, CA (US); Matthew Alexander Griebel, San Luis Obispo, CA (US); Robert J. DeRohan, San Luis Obispo, CA (US); Scott Garrett, San Luis Obispo, CA (US)

(72) Inventors: Nickolas Butler, San Luis Obispo, CA (US); Matthew Alexander Griebel, San Luis Obispo, CA (US); Robert J. DeRohan, San Luis Obispo, CA (US); Scott Garrett, San Luis Obispo, CA (US)

(73) Assignee: The Quality of Life Plus (QL+) Program, McLean, VA (US)

(21) Appl. No.: 13/447,098

(22) Filed: Apr. 13, 2012

Related U.S. Application Data
(60) Provisional application No. 61/475,186, filed on Apr. 13, 2011.
(60) Provisional application No. 61/476,316, filed on Apr. 17, 2011.
(60) Provisional application No. 61/480,964, filed on Apr. 29, 2011.
(60) Provisional application No. 61/492,584, filed on Jun. 2, 2011.
(60) Provisional application No. 61/492,432, filed on Jun. 2, 2011.
(60) Provisional application No. 61/492,501, filed on Jun. 2, 2011.
(60) Provisional application No. 61/501,035, filed on Jun. 24, 2011.
(60) Provisional application No. 61/503,961, filed on Jul. 1, 2011.
(60) Provisional application No. 61/569,273, filed on Dec. 11, 2011.
(60) Provisional application No. 61/569,274, filed on Dec. 11, 2011.
(60) Provisional application No. 61/579,926, filed on Dec. 23, 2011.

(51) Int. Cl. A61F 2/78

(52) U.S. Cl. CPC A61F 2/7812
USPC 623/36

(57) ABSTRACT

Prosthetic attachment techniques, systems, and apparatus are described including those having so called Chinese Finger Trap features.

### RELATED APPLICATIONS

This application claims the benefit of U.S. Provisional Application No. 61/475,186 entitled “Prosthetic Attachment Techniques” filed 13 Apr. 2011; this application is related to U.S. Provisional Application No. 61/452,618 entitled “Obstacle Detection for Visually Impaired Persons” filed 14 Mar. 2011; U.S. Provisional Application No. 61/453,040 entitled “Obstacle Detection for Visually Impaired Persons” filed 15 Mar. 2011; U.S. Provisional Application No. 61/476,316 entitled “Polygrasp Prosthetic Hand, Dual Mode Actuation, and Analog Myoelectric Interface” filed 17 Apr. 2011; U.S. Provisional Application No. 61/480,964 entitled “Blind Driver Challenge” filed 29 Apr. 2011; U.S. Provisional Application No. 61/492,584 entitled “Wheelchair Transfer Assist” filed 2 Jun. 2011; U.S. Provisional Application No. 61/492,432 entitled “Assistive Stair Climbing Device” filed 2 Jun. 2011; U.S. Provisional Application No. 61/492,501 entitled “Technological Advancement for Vehicles Operable by the Visually Impaired” filed 2 Jun. 2011; U.S. Provisional Application No. 61/492,584 entitled “Wheelchair Transfer Assist” filed 2 Jun. 2011; U.S. Provisional Application No. 61/501,035 entitled “Device for Assisted Entry For An Excavator” filed 24 Jun. 2011; U.S. Provisional Application No. 61/503,961 entitled “Prosthetic System for Foot, Ankle, and Tibia”” filed 1 Jul. 2011; U.S. Provisional Application No. 61/569,273 entitled “SCUBA Prosthesis” filed 11 Dec. 2011; U.S. Provisional Application No. 61/569,274 entitled “Edge Assist Hockey Prosthesis” filed 11 Dec. 2011; U.S. Provisional Application No. 61/579,926 entitled “PROSCUBA: Prosthetic SCUBA Leg filed 23 Dec. 2011; the entire contents of all of which application are incorporated herein by reference.

### BACKGROUND

present disclosure is directed to apparatus and methods that can provide prosthetics with suitable functionality to users, facilitating increased quality of life. A prosthesis can provide an artificial limb that simulates the function and natural feel of the replaced limb.

Many individuals have lost a limb for various reasons including war, accident, or disease. In most instances these individuals are not only able to live relatively normal lives, but physically active lives as well. Often times, these individuals are aided in their everyday lives by a prosthetic limb.

### SUMMARY

Aspects of the present disclosure are directed to prostheses that have a number of technical features that provide advantages or innovations compared to previous prosthetics. Prosthetic attachment techniques, including apparatus and methods, are disclosed, that simulate the function and natural feel of a replaced limb, and which are reliable, cost-effective, and robust. These innovations can present several advantages over existing market devices. The advantages can include any of the following, in any combination: increased attachment force, adaptable socket fit to accommodate swelling and shrinking of a residual limb (e.g., leg or arm), management of moisture accumulation from sweat, improved patient comfort through management of temperature, reduction in skin breakdown and resulting sores, reduction in pain caused by the socket, increased range of motion, and the combination of running and swimming prostheses.

### BRIEF DESCRIPTION OF THE DRAWINGS

The drawings disclose illustrative embodiments. They do not set forth all embodiments. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it refers to the same or like components or steps.

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIG. 1 is a photograph/picture of an example of a residual leg sleeve.

FIG. 2 is a photograph/picture of a rotating swim/run ankle adapter shown attached to prosthetic foot in a) normal walk/run position, and b) 70-degree swimming position.

FIG. 3 is a photograph/picture a prototype of lower-profile adapter in normal position (left) and swimming position (right).

FIG. 4 is a photograph/picture a machined adapter in normal position (left) and swimming position (right).

FIG. 5 shows two view of a so-called “Chinese Finger Trap”; and

FIG. 6 is a photograph showing a sleeve including a “Chinese Finger Trap” weave connected to a prosthetic leg.

While certain embodiments are depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

Aspects of the present disclosure are directed to prostheses that have a number of technical features that provide advantages or innovations compared to previous prosthetics. The advantages can include, but are not limited to the following, in any order and any combination and/or sub-combination: increased attachment force, adaptable socket fit to accommodate swelling and shrinking of a residual limb (e.g., leg or arm), management of moisture accumulation from sweat, improved patient comfort through management of temperature, reduction in skin breakdown and resulting sores, reduction in pain caused by the socket, increased range of motion, and the combination of running and swimming prostheses. Exemplary embodiments are directed to prosthetic attachment techniques, including apparatus and methods, are disclosed, which are reliable, cost-effective, and robust.

The proposed innovation presents several advantages over existing market devices. The advantages include: increased attachment force, adaptable socket fit to accommodate swelling and shrinking of the residual limb, management of moisture accumulation from sweat, improved patient comfort through management of temperature, reduction in skin breakdown and resulting sores, reduction in pain caused by the socket, increased range of motion, and the combination of running and swimming prostheses. Each advantage is discussed in further detail below.

### Increased Attachment Force

A technical feature according to the present disclosure provides increased attachment force to a patient/user. The technical features, or means for attachment, can prevent or facilitate prevention of a prosthetic device from slipping off the patient's residual limb, e.g., arm or leg.

Functioning similar to a Chinese finger trap produced as a kid's toy, the weave-braid design allows the Chinese finger trap to increase its attachment force in response to increased tension. Therefore in situations such as free-fall or applied forces from high-speed travel, the Chinese finger trap can grip the residual limb (e.g., leg) tighter. This design may be a secondary attachment system/feature, in addition to one or more other attachment features or means for attachment, e.g., a liner and/or ratcheting band or belt.

The Chinese finger trap can facilitate a snug fit between the residual limb/appendage (e.g., a leg) and the related liner even during applied tension and/or compression. Therefore the effect of “pistoning” (where the residual limb, e.g., leg, moves up and down vertically in the socket during activity) is practically mitigated or eliminated.

### Adaptable Socket and Attachment Fit

Especially during the first eighteen months after amputation, the residual limb swells and shrinks throughout the day due to the displacement of soft tissue. Current prosthesis designs are static and do not accommodate for any changing of shape or size. A technical feature of the present disclosure can incorporate either or both of two mechanisms or means which can allow for quick, adaptable adjusting of the prosthesis to maintain the desired fit at all times.

One mechanism, or “means for attachment” (alternatively “attachment means”) may include a ratcheting buckle and strap and/or a Chinese finger trap for attachment to a residual limb, e.g., the upper thigh of the residual leg. When both are used together, the ratcheting buckle may be connected to or built into the Chinese finger trap. In use, the strap is fed through the buckle and is tightened by pressing on the ratcheting buckle. Each press of the buckle can tighten the strap one step. Pulling on the buckle quickly can loosen the strap. Therefore the patient can quickly tighten or loosen the Chinese finger trap attachment by simply pressing the buckle, without needing to remove any portion of the prosthesis. In exemplary embodiments, a snowboard binding may be used for the ratcheting buckle. For example, a buckle and strap from a Freestyle snowboard binding made by Burton may be used. Other suitable ratcheting straps and buckles may be used. For example, suitable small metal ratchet buckles and ladder straps, (e.g., with dimensions of 15/16″×7 13/16″ or 198 mm×23.5 mm) are available for sale at Snowboards-for-sale.com, Inc., 1001 Avenida Pico, Suite C 528, San Clemente, Calif. 92673. Any suitable materials may be used for the ratchet buckles. Examples include but are not limited to suitable metals, metal alloys, and/or hard plastics/polymers. Any suitable materials may be sued for the straps. Examples of suitable strap materials include but are not limited to: Traditional TPU ankle straps sewn on top of die cut or molded EVA; Traditional over the top TPU toe straps sewn on top of die cut EVA; Lycra covered EVA; Perforated and non-perforated leather covered EVA; and/or Suede leather covered EVA.

Another mechanism, or “means for fit adjustment” (alternatively “adjustment means”) may be used to address swelling and shrinking in the area of the socket of the prosthetic. A means for adjustment can include an inflatable bag system that can be attached to the liner used for the prosthetic. A similar concept is proven and used in basketball shoes, baseball gloves, and ski boots. One or more inflatable bladders can be designed (placed or positioned) around the outer circumference of the liner, and can be pumped with air using a small button-like pump built into the tongue of the liner. When the residual leg shrinks, the bladders of the liner can be quickly inflated with a few presses of the pump. Since the socket is a fixed size and shape, the inflation of the bladders will push radially inward on the liner, causing the diameter of the liner to decrease. This conformably tightens the fit of the liner around the shrunken residual leg. If the limb (e.g., leg) swells, the bladders can be deflated by pressing the release valve also on the tongue of the liner. This system allows the patient to quickly adjust the fit of the liner around the limb and inside the socket in a matter of seconds.

### Sweat/Moisture Management

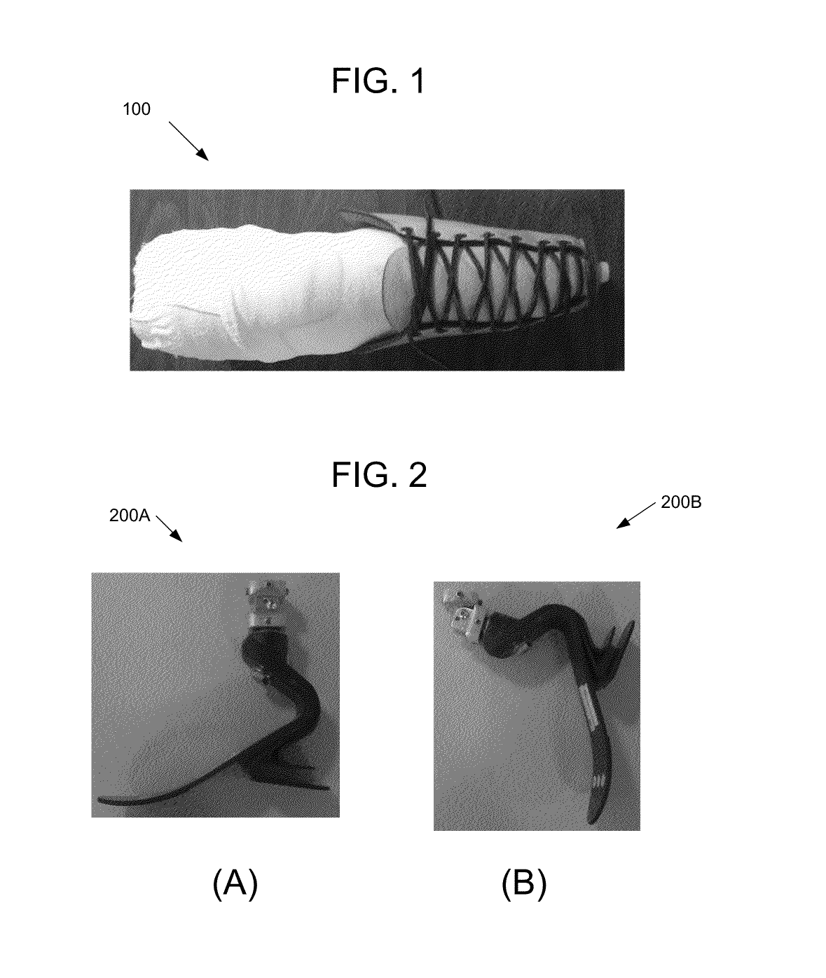

A technical feature of the present disclosure can provide advantages for sweat management, which is an important issue attributed to several other additional complications. This technical feature may be referred to as a moisture (or sweat) management mechanism, system, or means for moisture management (or sweat management), or moisture (or sweat) management means. Existing, patented technology was leveraged in partnership with Drymax® from their athletic socks. QL+ co-designed a sleeve with Drymax® specifically for use with the prototype prosthetics shown in FIG. The sweat-removing sleeve can be worn directly over the residual limb, e.g., leg (FIG. 1), and manages moisture by shunting sweat away from the skin. FIG. 1 shows a Drymax residual leg sleeve demonstrated being worn directly over skin, then inserted into leather liner.

The design leverages existing technology from Drymax athletic socks, proven to stay up to 25 times drier than leading competitor wicking socks. There is a noticeable difference between wicking fibers and Drymax® technology. The wicking fibers are hydrophilic (water-attracting) and immediately absorb sweat from the skin. However, the moisture stays in the wicking fiber layer, and remains in constant contact with the skin. Failure to remove the moisture away from the skin is undesirable because the moisture keeps the skin wet. Wet socks stick to the skin, increasing the friction between the two which leads to skin breakdown and sores. Over time, the moisture saturates the wicking fibers, upon which moisture continues to build up against the skin, thus rendering the wicking fiber sock useless.

Unlike the wicking sock, the Drymax sock is designed with two different fiber technologies that are interwoven to form a dual-layer sock. Moisture will not adhere to the hydrophobic (water-repelling) inner layer. Instead, moisture continues to travel and is squeegeed off the skin to the outer hydrophilic layer. Doing so keeps the skin dry, thus preventing the leg from slipping out of the device. Once moisture is transported to the outer layer of the sleeve, moisture will be expelled from the outer layer of the Drymax® sleeve into the liner. The breathable leather liner prevents sweat from pooling up inside the socket. Small holes in the bottom of the liner allow the moisture to drain out, effectively removing all sweat. This eliminates the “pistoning” effect experienced with the widely used silicone liners. Other suitable materials may be used in addition to or substitution for Drymax®. Examples include but are not limited to polypropylene fabrics.

### Temperature Management

A further technical feature of the present disclosure is temperature management or regulation for a prosthetic. This feature may be referred to as a temperature management mechanism or means for temperature management (or management means). The sweat management Drymax® residual leg sleeve and breathable liner mentioned above directly contribute to a reduction of heat experienced around the residual leg. The Chinese finger trap's open, mesh-like design also allows constant air flow to the residual leg, keeping it cool and dry. Since the Chinese finger trap can be attached at a desired place on a patient's limb, e.g., high on the thigh patient's residual leg, there is no need to cover the entire leg with a long liner or other wrapping. Thus, the main source of heat and discomfort experienced with current prosthesis attachment methods can be mitigated or eliminated.

### Skin Breakdown and Sores

The breakdown of skin and resulting sores occurs when the residual leg rubs against the liner and socket, mainly due to improper fit. Several of the design implications or technical features described above also address skin breakdown and sores. The improved fit with the Chinese finger trap can prevent the residual leg from moving and rubbing against the liner. The adaptable socket can keep the fit tight even during swelling and shrinking of the residual limb. The breathable liner and/or Drymax® sleeve can prevent moisture accumulation and keep the skin dry, which immensely reduces the chance of skin breakdown or sores because bacteria growth requires damp, warm, dark areas. The Drymax® residual leg sleeve can also contain copper and other substances which are proven to reduce bacteria and fungus growth, thus decreasing the chance of infection and sores. Furthermore, extra padding built into strategic areas of the leather liner prevents skin breakdown in the bony areas where patients have commonly lost the soft tissue in their residual limb.

Reduced Pain from Socket

Because a liner used in accordance with the present disclosure can be custom-designed to the patient's leg, it also has the advantage of reducing pain and irritation with specifically placed padding. As mentioned above, the loss of soft tissue leaves several common bony areas of the residual limb which contact the hard socket and cause pain. Current silicone liners are too soft, and do not provide sufficient cushion between the residual leg and the socket. A leather can liner provide a solid outer surface which mates with the inside of the socket. Padding built into the inside of the leather liner can provide a soft, compressible cushion surrounding the residual limb, e.g., arm or leg. Therefore pain can be greatly reduced, and the liner integrity is not compromised.

Increased Range of Motion

Previous issues of inhibited knee flexion (mainly experienced with below-the-knee amputees) were also solved with the new design. The leather liner is shaped such that the back of the knee if uncovered. This allows full range of flexion, compared to the greatly limited motion with current silicone liners and multiple sleeves. The material of the Chinese finger trap is flexible, and also does not inhibit any range of motion.

### Combination of Running and Swimming Feet

Additional advantages accompany the swim/run ankle adapter component. The adapter allows the patient to pivot the foot into a desired position, e.g., seventy-degree angle position, to be used during swimming as shown in FIG. 2, in views (A) and (B), which show a rotating swim/run ankle adapter attached to a prosthetic foot in two positions: (A) a walk/run position, and (B) a 70-degree swimming position.

Such multi-position functionality can eliminate or reduce the need to carry two separate prosthetic legs for swimming and running/walking as required currently amputees. The quick pin-mechanism allows the patient to switch easily between swimming and running positions in a matter of seconds, as compared to several minutes when previously swapping out the entire prosthetic leg. This also eliminates the need to carry several tools which were previously required to remove and attach different prosthetic feet and legs.

Parts and materials of prosthetics according to the present disclosure may be off-the-shelf components. Therefore may a particular material or component be discontinued by a certain vendor, the parts can simply be ordered through another supplier. The swim/run ankle adapter can be a custom engineered design, e.g., fabricated in-house. Any machine shop may be consulted to fulfill the machining requirements.

Prostheses according to the present disclosure, including the implemented prototype, can be considered to be revolutionary because they address five of the most common problems reported with existing prosthetic devices: socket fit as the limb swells or shrinks, temperature (heat) that produces discomfort, sweating and moisture accumulation, skin breakdown or sores, and pain caused in the area of the socket. Design aspects such as the Chinese finger trap, the adjustable ratcheting buckle for attachment, the inflatable bladders incorporated into an alternate-material liner, and the universal ankle adapter are all things that have never been applied to prostheses before.

Other commercial products sometimes present new improvements, but only attempt to address one problem at a time. For example, companies such as Otto Bock introduced a vacuum system to improve the attachment of the prosthesis. However this design relies on suction, which requires the residual leg to be completely encapsulated in the device. While this design claims to improve attachment, it makes other issues worse such as sweat accumulation, skin breakdown, and discomfort due to heat.

Another example product is the “volume sleeve” which is manufactured by several prosthesis companies. Nylon or polyester-like sleeves of varying thickness are marketed to address the socket fit problem during swelling and shrinking of the residual limb. Thicker sleeves are to be added over the residual limb when it shrinks, thus making up for the lost volume. Sleeves can be removed as the limb swells. This method does address the socket fit problem, but introduces additional problems of greater importance. Adding or removing these sleeves requires complete dis-attachment from the prosthesis which can become burdensome and unpractical throughout the day. Again, this design only addresses one challenge while compounding other problems such as sweat and temperature.

A leg prosthesis that switches into a swimming position is not a new concept. In fact, swim feet are quite common today. However current swim feet are only optimized for swimming, and are not designed to withstand the forces and wear from everyday walking and running use. Thus the user must immediately switch back to their normal prosthesis after they get out of the water with the swim foot. Because our swim/run ankle adapter is designed to replace the existing static pyramid ankle adapter, it can be used to turn any existing prosthetic leg assembly into a multi-purpose leg. Our universal adapter fits all standard pyramid male and female adapters, which is the most commonly used component. This avoids having the patient switch to a completely new leg or foot design.

Each of the design components in the proposed innovation work in conjunction to improve the quality of the overall prosthetic device. There exists no product on the market today which effectively addresses all five of the most common problems reported from amputees. Our system addresses all five of these problems through an innovative design, and also incorporates several additional advantages.

### Combination of Running and Swimming Feet

A swim/run ankle adapter according to the present disclosure allows the client to pivot the foot quickly by pushing a button or removing a pin, and lock it into a specified angle, e.g., a seventy-degree angle position to be used during swimming.

Any desired angle may be specified, e.g., an angle within the range of 50-80 degrees inclusive of the end values. A further example would be 65 degrees, 72 degrees, 75 degrees, etc. Other numerical vales may of course be selected. This can eliminate the need to carry two separate prosthetic legs for swimming and running/walking as required previously by the client. Further, the positioned may in exemplary embodiments be locked at any position or selected position (angles) within the overall range of motion (e.g., within a 30-degree arc).

The quick pin-mechanism can allow the client or user to switch easily between swimming and running positions in a matter of seconds, as compared to minutes when previously swapping out the entire prosthetic leg. This also eliminates the need to carry several tools which were previously required to remove and attach different prosthetic feet and legs.

### Universal Adapter

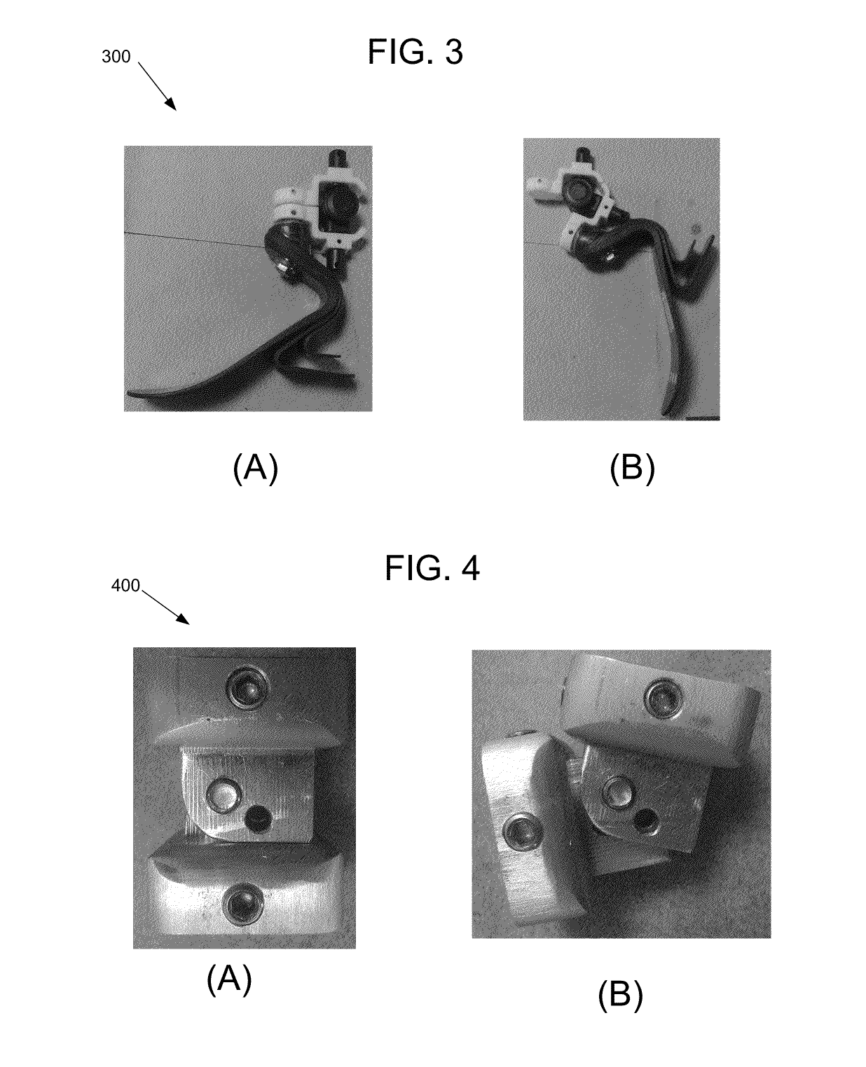

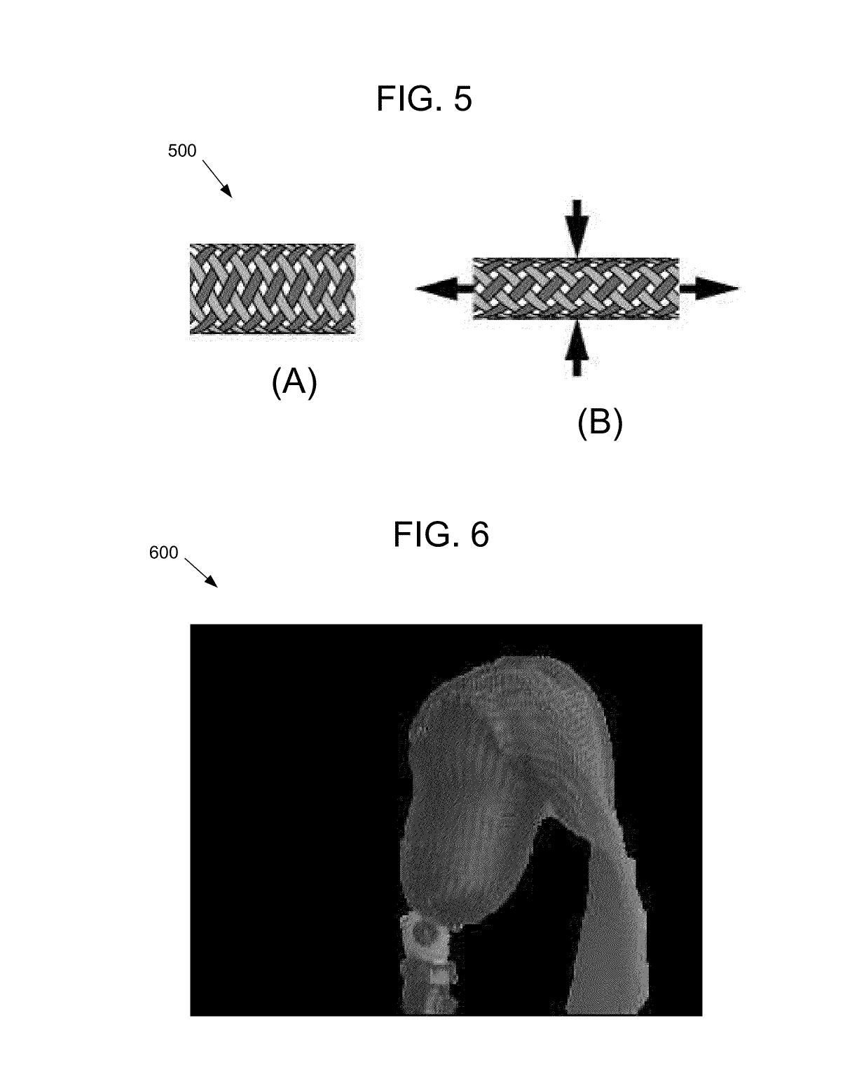

The ankle adapter is designed such that it fits most standard prosthetic leg devices. It simply replaces the static ankle which exists on most prosthesis assemblies. The pyramid shaped mating component of most existing prosthetic components fits into either side of the adapter. It is then tightened and adjusted using the four set screws in the same manner that a typical prosthetic foot is attached and fitted to the leg, as shown in FIG. 3, in views (A) and (B).

FIG. 4 shows a machined adapter in normal position (left) and swimming position (right). Changing positions is done by removing and re-inserting a pin.

Standard commercially available adapters or mates may be used. For example, standard adapters from the Otto Bock company, e.g., doing business as Otto Bock Health Care, having a place of business at Two Carlson Parkway North, Suite 100, 55447 Minneapolis, Minn. USA, may be used. Suitable adapters include, but are not limited to, Otto Bock Tube Adapters models 2R37, 2R38, 2R50, 2R49, 2R2, 2R3 and 2R38=10°, Otto Bock Tube Clamp Adapter, and/or Otto Bock Double Adapter. Any suitably strong and durable material(s) may be used for an adapter.

### Chinese Finger Trap:

A prototype of a Chinese finger trap was made out of a carbon fiber braid, and used for the prosthesis prototype. The carbon fiber braid was purchased from an outside vendor who makes it for a sleeve to cover cables. For the prototype, a 6″ diameter×3′ length sleeve was ordered/purchase, and then “cut” to length using a hot knife (this cauterizes the edges so it doesn't fray). For the prototype, braided carbon fiber biaxial sleeve—roll—6″—3 yard Roll (Part Number 2635-B) was used from vendor Fibre Glast.

As far as a braid that may be used for the Chinese finger trap, a biaxial braid is preferred. Such a braid can provide a sleeve that will both lengthen during tension (reducing its diameter) AND shorten past its resting length during compression (increasing its diameter). Other weaves and braids may only do one of these. A triaxial braid may be used though it may not necessarily perform as well as a biaxial braid. A triaxial braid may have a bit more stability than the biaxial. Compared with biaxial braids, a triaxial braid may be: 1) much harder to find “off-the-shelf”; 2) worse in compression properties than biaxial, which may lead to a concerned that the OD may not increase enough to be able to slip the thing over an appendage (e.g., leg) when putting it on. The general category of braids used for the Chinese finger trap can be cylindrical, helically wound braid, under which category both the specific biaxial and triaxial braids fall.

A suitable Chinese finger trap could be made out of any polymer. A significant factor to the functionality is the braid, not the material. For example, nylon, propylene, and other polymers may be used. Other materials may of course be used for a Chinese finger trap, e.g., leather strips or chords, Kevlar, Spectra, natural fibers, with or without polymer coatings, etc.

Calculations were performed for the design of the Chinese finger trap feature of the prototype. For the calculations of the Chinese finger trap, general statics/dynamics were considered/evaluated to get the tension forces that were anticipated to be applied to it during usage, such as when the client is in free fall or high-speed boating. The diameter of the client's residual leg, where the Chinese finger trap would be attached, was taken into account.

For some embodiments/applications, such as the prototype, a ratcheting strap may be used as part of an attachment means used for a prosthetic. For some applications/embodiments, the Chinese finger trap can serve as a secondary or backup attachment mechanism/means. Accordingly, it may be desirable for the Chinese finger trap to remain slightly loose during low-exertion activities such as sitting or walking—simply for comfort reasons. For some applications and implementations, it may be desirable to avoid a bunch of slack before the Chinese finger trap engages by becoming taught. To that end, an estimate can be made of the distance in which the Chinese finger trap would stretch from resting position until fully taught. This may be a small distance, e.g., around 0.25″. With this information, a calculation can be made of how long to cut the Chinese finger trap (length being the distance from the location of attachment on the residual limb (e.g., leg), to the location where it may be attached, e.g., to the socket. The longer the sleeve (attached lower on the socket), the more stretching it allows.

While only one strap, configured to be placed or positioned around the top of the thigh for attachment of the prosthetic to the limb, was used for the prototype, multiple straps may be used and these may run either vertically or circumferentially, or both (or in other orientations, e.g., diagonally, obliquely), for more precise fit adjusting. The straps may be built into the Chinese finger trap, or may simply be used on top of or externally to the Chinese finger trap feature.

### Swim/Run Ankle Adapter

A prototype of a swim/run (two mode, or two position) ankle adapter was made out of aluminum for the prototype prosthetic/prosthesis. The type of alloy was unknown but thought to be 6061. Any suitable aluminum alloy with any type of surface/hardness treatment may be used. Titanium is a preferable material for the swim/run adapter) in order to reduce weight. It would be possible to use any type of metal or metal alloy for the adapter; a suitably strong polymer could also be used. Corrosive resistance is a desirable or key attribute for selecting the material of the adapter. Other suitable materials include, but are not limited to, Kevlar (registered trademark of DuPont for a para-aramid synthetic fiber), Delrin (proprietary acetal resin of DuPont), and their generic equivalents. Suitable steels may be used, of any alloy type and composition. Exemplary steels include stainless steels such as 302 or 316L alloys. Any alloy of titanium may be used; exemplary alloys include 6AL-4V, 3AL-2.5V, and 3AL-2V.

The adapter for the adapter prototype was machined from stock aluminum bar. Pins and nuts/bolts were purchased (standard ANSI sizes) through McMaster-Carr, but suitable ones may be bought at any hardware store. The pin used was a McMaster-Carr P/N: 98306A104 (Clevis Pin).

A second prototype design was made for the adapter component, which utilized a push-button on the side, instead of the pin, for locking and unlocking the prosthetic foot/lower leg in the two positions. A positional-hinge was ordered off McMaster for a proof-of-concept prototype. Other embodiments could be machined for the final product. As mentioned above, titanium is preferable, with perhaps some other % of metal mixed for corrosion resistance.

### Prosthetic Foot

For an implemented prototype, a carbon-fiber based commercially available prosthetic foot was utilized. This was a stock, commercial off the shelf (COTS) component referred to as the Renegade® model from manufacturer Freedom Innovations, LLC, having a place of business at 30 Fairbanks, Suite 114, Irvine, Calif. 02618. This foot was used as purchased—showing a main advantage of the design, i.e., that existing prosthetic feet may be used with no modifications.

Any other suitable design for a prosthetic foot may be used for apparatus and methods in accordance with the present disclosure. For example, suitable prosthetic feet can include but are not limited the following made commercially available by Freedom Innovations, LLC: Renegade® MX, Thrive TM, Runway®, Silhouette®, Silhouette® LP, Silhouette® VS, Silhouette® LP-VS, Sierra®, Highlander®, Defender®, Senator®, LP Symes, Nitro Running Foot, Slalom Ski Foot, Freestyle Swim, and Promenade™. The following patents, the entire contents of all of which are incorporated herein by reference, describe suitable prosthetic feet for use with embodiments described herein: U.S. Pat. No. 7,419,509; U.S. Pat. No. 7,462,201; U.S. Pat. No. 7,520,904; U.S. Pat. No. 7,572,299; U.S. Pat. No. 7,618,464; U.S. Pat. No. 7,686,848; U.S. Pat. No. 7,727,285; and, U.S. Pat. No. 7,824,446. Other suitable prosthetic feet include, but are not limited to, the following as made commercially available by the Otto Bock company: Carbon feet including the Trias® foot, the C-Walk foot, the Axtion® foot, the Axtion DP foot, the Advantage DP2 foot., the Spinglite II foot; Dynamic feet, including the Dynamic Motion Foot, and the Dynamic Foot; the 1 A30 Gressinger Plus Foot; the 1M10 Adjust Foot; and the Sach Foot. Other prosthetic feet may of course be used, including ones with telescoping shock absorbing features add mechanism.

### Liner

The liner used for the prosthetic can be made of any suitable material. It is preferably made from leather, as was the case for the prototype prosthetic. Other suitable materials may me used in addition with or substitution for leather. Examples include, but are not limited to, synthetic leather such as available under the trade name Alcantara™. Neoprene or other kinds of synthetic rubber may also be used.

The layout, sizes, padding, and features may be tailored for a specific individual. The liner for an implemented prototype was made from a local leatherworker in Morro Bay, Calif. at “The Leather Shop.”

The objective of the project was to improve upon current lower leg prosthetic designs to develop a new prosthetic to be used by the client in combat. Upon a visit to Norfolk, Virginia and meeting with the client, the team learned the requirements for his new prosthetic. The requirements were discussed agreed upon by the client and design team. They are the specifications that the newly designed prosthesis will preferably adhere to (or surpass) in order to be considered acceptable by Navy SEAL standards. The following are the requirements in order of importance:

Does not detach once attached to client.

Wick away sweat to prevent pistoning and irritation.

Properly conform to client's leg to minimize pain and irritation.

Enable minimal to no limitations of knee flexion.

Include a mechanism for switching the foot between walking/running and swimming positions.

Withstand the forces generated by the weight of client and his gear, plus an additional 100 pounds.

As explained by the client, the largest issue with the current prosthesis is the attachment mechanism. Thus, specifications one through four are the primary focus for this new design. Both specifications five and six are tied to additional functionality and together were considered the secondary focus. The assigned engineering team aimed to combine both new and existing designs into a single prosthetic leg to address each of these presented challenges.

### Conceptual Design

Several ideas were conceived during the brainstorming segment of the project. A brief description of the highly-considered ideas can be found below.

Inflatable Liner/Socket

Several inflatable bags built into the liner and/or socket can be pumped with a compressible fluid (such as air) to adjust the tightness of attachment onto the residual leg.

This design was inspired by Reebok's PUMP basketball shoes that debuted in the 1980's. Similar to the shoes, a thumb-sized built-in pump could be used to inflate the built-in bags (thus, tightening the liner), or deflate the liner. The inflatable bags would conform around the residual leg creating a snug fit, thus reducing pain and irritation. The bags could also be easily adjusted throughout the day to address the constant loss of volume in the residual leg.

### Elevated Vacuum

Preliminary research indicated that switching the client from a suction liner system to an elevated vacuum system may add attachment force. Such vacuum liners are sold by many leading prostheses manufactures.

### Secondary Suspender

If the primary attachment system (such as the suction liner) were to fail, a secondary attachment system (such as a belt or suspender attached to the prosthesis) would keep the prosthesis from slipping completely off the client's leg. One example is depicted below in FIG. 8, below.

### Leather Strapping System

Silicone does not breathe, and thus does not allow any sweat to drain. Creating an alternative liner out of leather, or series of straps to replace the liner would give the leg some degree of breathability. Another client of QL+ currently uses a leather strap attachment around his residual hand, and claims the leather to manage sweat better than other materials.

### Liner Modifications

Several modifications could be made to the client's existing liner to improve sweat management. Such modifications include adding drainage channels (FIG. 4), and one-way check valves at the bottom of the liner that would expel sweat when the client walks.

Micropores could also be etched through the liner to allow sweat to drain out the liner as it would a sock or shirt.

### Chinese Finger Trap for Attachment

When a Chinese finger trap is pulled in tension, its diameter decreases and it provides a strong inward force that locks down on the user's finger, effectively preventing it from being removed, as shown in FIG, 5, views (A) and (B).

A similar design could be applied to an attachment system. One end of a larger Chinese finger trap-like sleeve would be placed over the upper thigh and the other end over the prosthesis. Therefore when the prosthesis begins to slip off the residual leg, it pulls on the Chinese finger trap which in turn tightens down on the residual leg. This tension would keep the device attached to the leg at all times.

### Pin-Vise Mechanism for Quick Swapping of Swim/Run Feet

The current attachment of feet requires the removal of four set screws. This requires the need to carry a separate tool, and also requires an extended period of time to swap out feet. A simpler mechanism, such as a pin vise could be quickly undone to speed up the removal and attachment of feet.

### Snowboard Strap and Buckle for Attachment

A strap and buckle could be built into the liner to allow for an adjustable attachment system. Throughout the day when the residual leg loses volume, the client could initiate a quick ratchet of the buckle with his hand that would tighten the strap around the top of the liner to prevent it from slipping off.

### Rotating Swim/Run Ankle

To address the need for carrying multiple feet for running and swimming activities, the adapter that holds the foot in place could be re-designed. The new ankle adapter would rotate so that the foot could be set into a desired position, e.g., 70 degree angle position, for swimming.

The Chinese finger trap 600 of FIG. 6 provides for the ability to provide increased attachment force, e.g., to a client. Such a design can prevent the prosthetic device from slipping off the client's residual leg. The weave-braid design allows the Chinese finger trap to increase its attachment force in response to increased tension. Therefore in situations such as free-fall or applied forces from high-speed travel, the Chinese finger trap will grip the leg tighter.

### Improved Sweat Management

In addition to attachment force, the design solution also provides advantages for sweat management.

The breathable leather liner prevents sweat from pooling up inside the socket. This in turn eliminates the “pistoning” effect experienced with the client's previous liner. Because the leather liner was custom-designed to the client's leg, it also has the advantage of reducing pain and irritation with specifically placed padding. The sweat-removing sleeve also solves this problem by shunting sweat away from the skin. Doing so keeps the skin dry, thus preventing the leg from slipping out of the device.

### Increased Range of Motion

Previous issues of inhibited knee flexion were also solved with the new design. The leather liner is shaped such that the back of the knee if uncovered. This allows full range of flexion, compared to the greatly limited motion with the client's previous silicone liner and multiple sleeves. The material of the Chinese finger trap is flexible, and also does not inhibit any range of motion.

### Combination of Running and Swimming Feet

Additional advantages accompany the swim/run ankle adapter component. The adapter allows the client to pivot the foot into a seventy-degree angle position to be used during swimming. This eliminates the need to carry two separate prosthetic legs for swimming and running/walking as required previously by the client. The quick pin-mechanism allows the client to switch easily between swimming and running positions in a matter of seconds, as compared to minutes when previously swapping out the entire prosthetic leg. This also eliminates the need to carry several tools which were previously required to remove and attach different prosthetic feet

The components, steps, features, benefits and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits and advantages. These also include embodiments in which the components and/or steps are arranged and/or ordered differently. For example, while descriptions herein of various features have been given in the context of use with a prosthetic leg or foot, technical features and aspects of the present disclosure may be directed to and encompass other prosthetics, for example, prosthetic arms and hands.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. All articles, patents, patent applications, and other publications which have been cited in this disclosure are hereby incorporated herein by reference.

What is claimed is:

1. A prosthetic attachment system comprising:
a woven sleeve adapted to fit an appendage; and
an attachment for connecting the woven sleeve to a prosthetic leg.

2. The prosthetic attachment system of claim 1, wherein the woven sleeve is configured as a Chinese finger trap.

3. The prosthetic attachment system of claim 1, wherein the attachment comprises a hinge configured to be set in a selectable one of two positions.
